(19) United States
(12) Patent Application Publication
(10) Pub. No.: US 2008/0027399 A1
(43) Pub. Date: Jan. 31, 2008

(54) ANTIMICROBIAL VASCULAR ACCESS DEVICE

(75) Inventors: Weston F. Harding, Lehi, UT (US); Glade H. Howell, Sandy, UT (US); Bryan G. Davis, Sandy, UT (US); Austin Jason McKinnon, Herriman, UT (US); Kelly D. Christensen, Centerville, UT (US); S. Ray Isaacson, Roy, UT (US); Wayne K. Rasmussen, Riverdale, UT (US); Christopher N. Cindrich, Draper, UT (US); Marty L. Stout, South Weber, UT (US)

Correspondence Address: David W. Highet;Becton, Dickinson and Company, (Metcalf Intellectual Property Law, LLC), 1 Becton Drive, MC 110, Franklin Lakes, NJ, 07417-1880, US

(73) Assignee: BECTON, DICKINSON AND COMPANY, Franklin Lakes, NJ (US)

(21) Appl. No.: 11/829,004

(22) Filed: Jul. 26, 2007

Related U.S. Application Data
(60) Provisional application No. 60/820,641, filed on Jul. 28, 2006.

(51) Int. Cl. A61M 25/00

(52) U.S. Cl. 604/265

(57) ABSTRACT

A medical device includes a body and an interior surface of the body that communicates with a fluid capable of delivering a pathogen to the interior surface. The medical device may also have an energy source coupled with the vascular access device that provides energy to the interior surface of the body to repress pathogenic activity. A method of repressing pathogenic activity in a vascular access device includes providing a body having an interior surface and energizing the vascular access device to repress pathogenic activity on the interior surface.

### RELATED APPLICATIONS

This application claims the benefit of U.S. Provisional Application No. 60/820,641, filed Jul. 28, 2006, entitled ANTIMICROBIAL VASCULAR ACCESS DEVICE, which is incorporated herein by reference.

### BACKGROUND OF THE INVENTION

The present disclosure relates to infusion therapy with antimicrobial vascular access devices. Infusion therapy is one of the most common health care procedures. Hospitalized, home care, and other patients receive fluids, pharmaceuticals and blood products via a vascular access device inserted into the vascular system. Infusion therapy may be used to treat an infection, provide anesthesia or analgesia, provide nutritional support, treat cancerous growths, maintain blood pressure and heart rhythm, or many other clinically significant uses.

Infusion therapy is facilitated by a vascular access device. The vascular access device may access a patient's peripheral or central vasculature. The vascular access device may be indwelling for short term (days), moderate term (weeks), or long term (months to years). The vascular access device may be used for continuous infusion therapy or for intermittent therapy.

A common vascular access device is a plastic catheter that is inserted into a patient's vein. The catheter length may vary from a few centimeters for peripheral access to many centimeters for central access. The catheter may be inserted transcutaneously or may be surgically implanted beneath the patient's skin. The catheter, or any other vascular access device attached thereto, may have a single lumen or multiple lumens for infusion of many fluids simultaneously.

The proximal end of the vascular access device commonly includes a Luer adapter to which other medical devices may be attached. For example, an administration set may be attached to a vascular access device at one end and an intravenous (IV) bag at the other. The administration set is a fluid conduit for the continuous infusion of fluids and pharmaceuticals. Commonly, an IV access device is a vascular access device that may be attached to another vascular access device, closes or seals the vascular access device, and allows for intermittent infusion or injection of fluids and pharmaceuticals. An IV access device may include a housing and a septum for closing the system. The septum may be opened with a blunt cannula or a male Luer of a medical device.

Complications associated with infusion therapy may cause significant morbidity and even mortality. One significant complication is catheter related blood stream infection (CRBSI). An estimate of 250,000-400,000 cases of central venous catheter (CVC) associated BSIs occur annually in US hospitals. Attributable mortality is an estimated 12%-25% for each infection and a cost to the health care system of $25,000-$56,000 per episode.

Vascular access device infection resulting in CRBSIs may be caused by failure to regularly clean the device, a non-sterile insertion technique, or by pathogens entering the fluid flow path through either end of the path subsequent to catheter insertion. Studies have shown the risk of CRBSI increases with catheter indwelling periods. When a vascular access device is contaminated, pathogens adhere to the vascular access device, colonize, and form a biofilm. The biofilm is resistant to most biocidal agents and provides a replenishing source for pathogens to enter a patient's bloodstream and cause a BSI.

Thus, what are needed are systems, devices, and methods to prohibit, limit, or otherwise eliminate vascular access device contamination to reduce the risk and occurrence of CRBSIs.

### BRIEF SUMMARY OF THE INVENTION

The present invention has been developed in response to problems and needs in the art that have not yet been fully resolved by currently available vascular access systems, devices, and methods. Thus, these developed systems, devices, and methods prohibit, limit, or otherwise eliminate vascular access device contamination to reduce the risk and occurrence of CRBSIs.

A medical device may be a vascular access device that includes a body and an interior surface of the body. The interior surface communicates with a fluid capable of delivering a pathogen to the surface. An energy source coupled with the vascular access device provides energy to the interior surface of the body to repress pathogenic activity. The medical device may have an interior structure in contact with the interior surface of the body, an exterior surface of the body, and an exterior magnet in contact with the exterior surface of the body. In this embodiment, the energy source is a magnetic force between the exterior magnet and the interior structure which may cause movement of the interior structure.

The interior surface of body may be formed from a degradable biocompatible material. The interior surface may be formed of an electrically conductive material, where the energy source is a battery that delivers electric current to the electrically conductive material. The interior surface may also include a heat conductor, where the energy source transfers heat to the heat conductor. The energy source may include an oscillator that causes rapid repetitive movement of the interior surface, a wave generator, or an antiseptic applicator. The energy source may also emit ultraviolet light on the interior surface of the body, deliver electric current to the fluid of sufficient magnitude and duration to repress pathogenic activity, and include anti-bacterial fluid applied to the interior surface of the body.

A method of repressing pathogenic activity in a vascular access device may include providing a vascular access device with a body having an interior surface and energizing the vascular access device to repress pathogenic activity on the interior surface.

Energizing may include actuating a magnet to disturb a pathogen residing on the interior surface, degrading the interior surface, supplying electric current to the interior surface, heating a heat conductor, vibrating the interior surface, generating and transmitting a series of waves against the interior surface, sterilizing the interior surface, emitting ultraviolet light towards the interior surface, and/or flushing the vascular access device with anti-bacterial fluid.

A medical device may also include means for accessing the vascular system of a patient and means for repressing a pathogen. The pathogen may reside within the means for accessing the vascular system of the patient. The means for repressing may include an energy source.

These and other features and advantages of the present invention may be incorporated into certain embodiments of the invention and will become more fully apparent from the following description and appended claims, or may be learned by the practice of the invention as set forth hereinafter. The present invention does not require that all the advantageous features and all the advantages described herein be incorporated into every embodiment of the invention.

### BRIEF DESCRIPTION OF THE SEVERAL VIEWS OF THE DRAWINGS

In order that the manner in which the above-recited and other features and advantages of the invention are obtained will be readily understood, a more particular description of the invention briefly described above will be rendered by reference to specific embodiments thereof which are illustrated in the appended drawings. These drawings depict only typical embodiments of the invention and are not therefore to be considered to limit the scope of the invention.

FIG. 1 is a perspective view of an extravascular system connected to the vascular system of a patient.

FIG. 2 is a side view of a vascular access device with a spin ring.

FIG. 3 is a cross section view of the vascular access device of FIG. 2 taken along lines A-A.

FIG. 4 is a cross section view of a vascular access device with a plastic ring.

FIG. 5 is a partial cross section view of the vascular access device of FIG. 4 taken along lines A-A.

FIG. 6 is a cross section view of a vascular access device with a degrading surface.

FIG. 7 is a partial cross section view of a vascular access device having an electrically conductive interior surface.

FIG. 8 is a cross section view of a septum of a vascular access device having a heat conductor.

FIG. 9 is a cross section view of a septum of a vascular access device having an oscillator.

FIG. 10 is a cross section view of a vascular access device coupled with a wave generator.

FIG. 11 is a side view of a vascular access device coupled with an ultrasonic wave generator.

FIG. 12 is a cross section view of a vascular access device coupled with a medicated blade plug.

FIG. 13 is a side view of a vascular access device with a medicated snap cap in open position.

FIG. 14 is a side view of the vascular access device of FIG. 13 with the snap cap in closed position.

FIG. 15 is a transparent side view of a vascular access device and a side view of a cap with an ultraviolet LED bulb.

FIG. 16 is a transparent side view of an alternate embodiment of the vascular access device and cap of FIG. 15.

FIG. 17 is a partial cross section view of a septum of a vascular access device coupled with an ultraviolet light source.

FIG. 18 is a perspective view of a vascular access device and an ultraviolet light isolator and exposer.

FIG. 19 is a transparent side view of an extravascular system having an ultraviolet light source coupled to a catheter and a vascular access device.

FIG. 20 is a transparent side view of a vascular access device coupled to an ultraviolet light source, which is in turn coupled to a catheter.

FIG. 21 is a cross section view of a sterilization cap coupled to a side view of a vascular access device.

FIG. 22 is a cross section view of a vascular access device, a catheter, and a grounded battery.

FIG. 23 is a cross section view of a vascular access device coupled with a flush pressure unit.

FIG. 24 is a more detailed cross section view of the vascular access device of FIG. 23.

### DETAILED DESCRIPTION OF THE INVENTION

The presently preferred embodiments of the present invention will be best understood by reference to the drawings, wherein like reference numbers indicate identical or functionally similar elements. It will be readily understood that the components of the present invention, as generally described and illustrated in the figures herein, could be arranged and designed in a wide variety of different configurations. Thus, the following more detailed description, as represented in the figures, is not intended to limit the scope of the invention as claimed, but is merely representative of presently preferred embodiments of the invention.

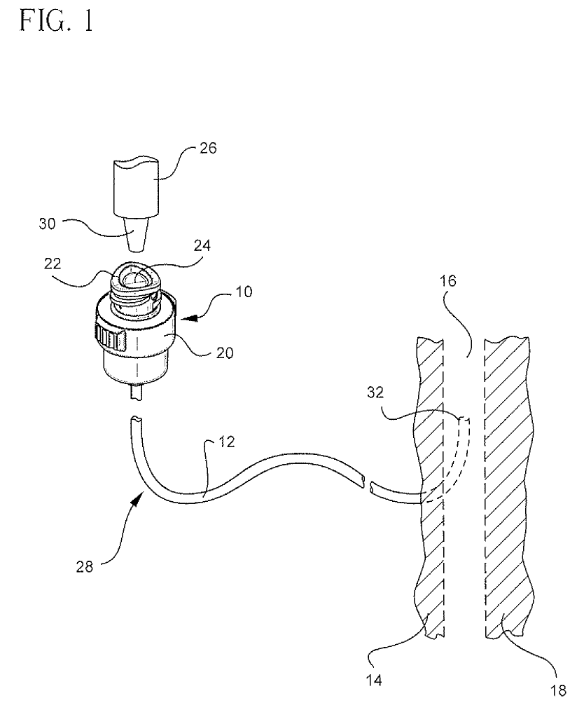

Referring now to FIG. 1, a vascular access device (also referred to as an extravascular device, intravenous access device, access port, and/or any device attached to or functioning with an extravascular system) 10 is used to introduce a substance via a catheter 12 across the skin 14 and into a blood vessel 16 of a patient 18. The vascular access device 10 includes a body 20 with a lumen and a septum 22 placed within the lumen. The septum 22 has a slit 24 through which a separate extravascular device 26, such as a syringe, may introduce a substance into the vascular access device 10.

The device 10 also includes an energy source (discussed with reference to the figures below) capable of repressing pathogenic activity within the vascular access device 10 and/or the extravascular system 28 to which the vascular access device 10 is connected. The energy source represses pathogenic activity to decrease the incidence of blood stream infections in patients to whom the vascular access device 10 or any other device on an extravascular system 28 is attached.

A pathogen may enter the device 10 or system 28 in any of a number of ways. For example, a pathogen may reside within the device 10 or system 28 prior to first use. A pathogen may also be introduced into the device 10 from the external surface of the device, the external surface of a separate device 26, and/or the surrounding environment when a structure such as a tip 30 of the separate device 26 is inserted into the device 10 through the slit 24 of the septum 22. A pathogen may be introduced within fluid that is infused into the system from a separate device 26. Finally, a pathogen may be introduced from a blood vessel 16 into the system 28 by entering through the end 32 of the catheter 12 during a blood draw or a period of blood reflux when the device 10 is in use.

As described throughout this specification, the energy source represses pathogenic activity by any one or combination of the following actions upon a pathogen: removing, dislodging, inhibiting growth, attracting to a location, repelling from a location, sloughing a pathogen and/or its attached surface or structure, degrading, frustrating, killing, heating, shearing, fragmenting, preventing growth or proliferation, radiating, electrifying, flushing, and/or any other similar process or action. Energy sources include electrical, ultrasonic, ultraviolet, magnetic, mechanical, nano vibrator, oscillator, white light, plasma, heat, e-beam, and other similar energy sources. Pathogens include any agent that causes a disease, infects a host, or otherwise harms or has the potential to harm a patient and/or host if received into the vascular system of that patient and/or host, including a pathogen, bacterium, parasite, microbe, biofilm, fungus, virus, protein feeding a pathogen, protozoan, and/or other harmful microorganisms and/or agents and products thereof. Finally, pathogenic activity includes the entry, travel, residence on a surface, growth, proliferation, organization, development, progression, and/or other similar activity into and within the device 11, system 28, and/or blood vessel 16.

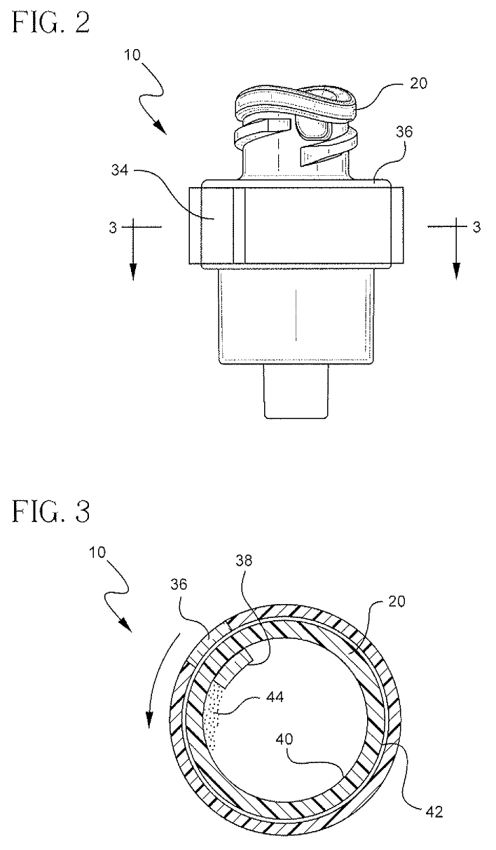

Referring now to FIG. 2, a vascular access device 10 includes a spin ring 34 located around the exterior surface of the device 10. The spin ring 34 includes a magnet 36 embedded within its body. The magnet 36 provides an energy source in the form of a magnetic force which is transferred through the body 20 of the device 10 to an interior structure 38 (FIG. 3).

Referring now to FIG. 3, a cross section of the device 10 of FIG. 2 is shown taken along lines A-A. As shown in FIG. 3, the exterior magnet 36 is in communication with the interior structure 38 by means of a magnetic force transferred between the exterior magnet 36 and the interior structure 38 through the body 20 of the device 10. The interior structure 38 may be a corresponding magnet, iron or other metal, and/or other magnetically conductive material capable of being influenced by magnetic force. The interior structure 38 operates under the influence of exterior magnet 36 to move or travel along an interior surface 40 of the device 10. Thus, as an operator moves or otherwise articulates the exterior magnet 36 around an exterior surface 42 of the device 10, the interior structure 38 moves in unison with the exterior magnet 36 to scrape, agitate, or otherwise disturb the interior surface 40 and any pathogen 44 which may reside thereon.

Thus, the embodiment of FIGS. 2 and 3 provides a vascular access device 10 with a magnetic energy force capable of cleaning, removing, disturbing, or otherwise agitating a pathogen 44 on an interior surface 40 of the device 10. In use, an operator can spin the spin ring 34 on the outside of the device 10 regularly. The magnet 36 on the spin ring drags or influences the magnetic scraper or interior structure 38 on the inside, cleaning any forming biofilm off of the interior surface 40. In an alternate embodiment, the spin ring 34 may include a weight on one end of the ring 34 such that any movement of the device 10 will cause the weight to pull the ring 34 in a direction under gravitational force and/or momentum, permitting the weight to rotate or spin the ring 34 in relation to the device 10. As the ring 34 automatically spins with movement of the device 10, the interior structure 38 will clean the interior surface of the device 10.

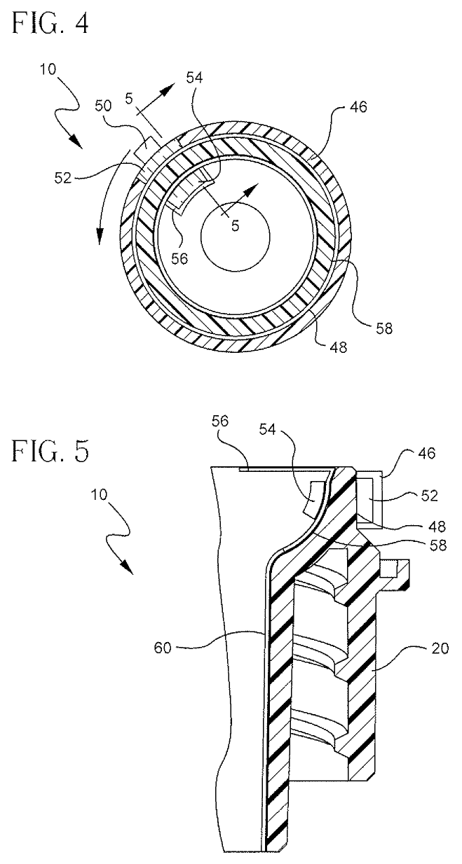

Referring now to FIG. 4, a cross section of a vascular access device 10 is shown. A ring 46 residing on an exterior surface 48 of the device 10 includes a knob 50 attached on the outer surface of the ring 46, and an exterior magnet 52 embedded within the plastic of the ring 46. Much like the embodiment described with reference to FIGS. 2 and 3, the exterior magnet 52 of the embodiment of FIG. 4 operates as an energy source providing magnetic force to influence an interior magnet 54. However, the interior magnet 54 of the embodiment of FIG. 4 includes an antimicrobial pad 56 surrounding the interior magnet 54. As the ring 46 is rotated around the exterior surface 48 of the device 10, the interior magnet 54 or other similar structure will follow the path of the exterior magnet 52 causing the antimicrobial pad 56 to swab an interior surface 58 of the device 10.

Referring now to FIG. 5, a partial cross section view of FIG. 4 shows the vascular access device 10 of FIG. 4. FIG. 5 shows the plastic ring 46 housing the exterior magnet 52 on the exterior surface 48 of the body 20 of the device 10. The exterior magnet 52 exerts magnetic force through the body 20 against a corresponding magnetic substance or interior magnet 54. The interior magnet 54 is attached to an antimicrobial wiper or antimicrobial pad 56. The antimicrobial pad 56 is formed in a horseshoe bend to correspond with the shape, size, and dimensions of the interior surface 58. Thus, the antimicrobial pad 56 which may also include an antimicrobial pad extension 60 along any portion of the interior surface 58, is structured to be able to clean any portion of the interior surface 58 when actuated.

Thus, the embodiment described with reference to FIGS. 4 and 5 provides an exterior magnet in contact with the exterior surface of the body of the vascular access device and a corresponding interior magnet and antimicrobial pad in contact with the interior surface of the body of the device. When the exterior magnet is moved, a magnetic energy source causes the interior magnet and corresponding antimicrobial pad to move, cleaning the interior surface of the device. In contrast to the embodiment described with reference to FIGS. 2 and 3, the embodiment described with reference to FIGS. 4 and 5 provides a chemical reaction intended to kill a pathogen upon contact of that pathogen with the antimicrobial pad. The embodiment described with reference to FIGS. 2 and 3, however, includes an interior structure or magnet that mechanically removes or otherwise damages or destroys a pathogen upon contact.

Embodiments alternate to those described with reference to FIGS. 2 through 5 may include other mechanical agitations on the interior of the device 10. For example, a pin or ball may reside within the device 10. An operator can shake the device 10 to bounce the pin or ball against the interior surfaces of the device 10, thus stirring the fluid therein.

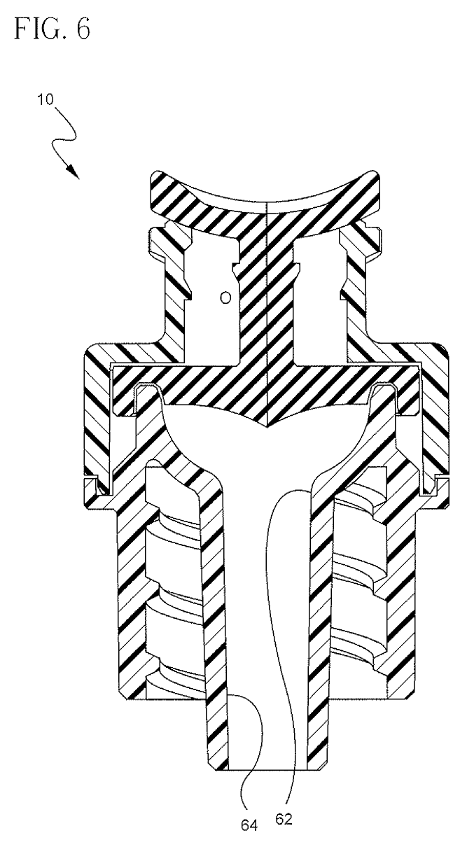

Referring now to FIG. 6, a vascular access device 10 includes a degrading surface 62 on the interior surface 64 of the device 10. The degrading surface 62 is formed from a degradable bio-compatible material that is soluble in saline or other common intravenous fluid that is infused into or through the device 10. The surface 62 may also be designed to work with a specific fluid having specific properties capable of degrading the surface 62 at a desired rate. The purpose of the degrading surface 62 is to prevent the formation of a biofilm by continuously shedding the interior surface 64 which comes into contact with infused fluid. The degrading surface 62 makes it very difficult for a pathogenic biofilm to grow on the surface 62, for protein to form on the surface 62, or for corresponding pathogens to be attracted to or subsequently bind with a protein layer formed on the surface 62. As the surface 62 degrades and sloughs from the interior surface 64, the surface, biofilm particles, proteins, and other pathogens, travel with the sloughed surface 62 along the fluid path of the device 10 and into the vascular system of a patient. Because pathogens have not resided on the surface 62 long enough to form a harmful bacterial culture and/or biofilm, their entry into the vascular system of a patient should cause less harm to that patient than an advanced bacterial culture would cause, and may cause no harm to the patient at all. The degrading surface 62 may be applied to any vascular access device 10 including an intravenous catheter.

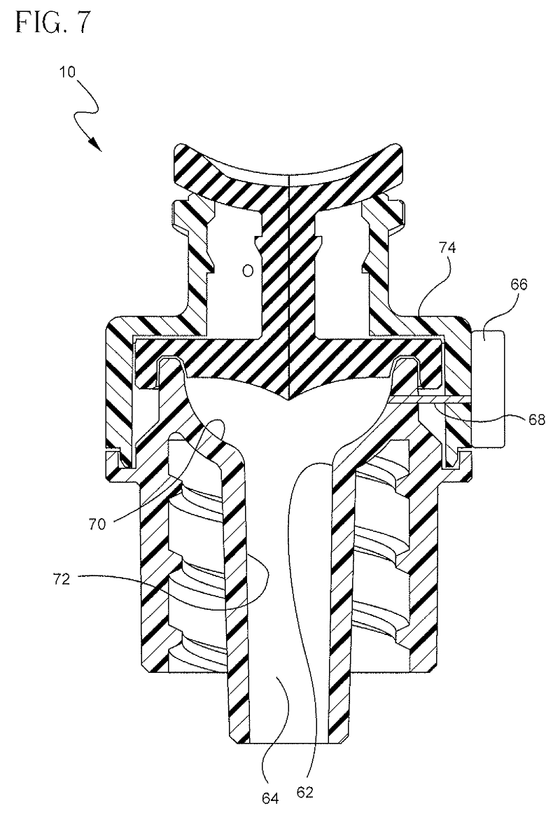

Referring now to FIG. 7, a partial cross section view of a vascular access device 10 reveals an energy source that includes a battery 66 that delivers electric current through a lead 68 to an electrically conductive material such as a metal coating 70 that resides on the interior surface 72 of the device 10. The battery 66 may reside on any portion of the device and will preferably be located on the exterior surface 74 of the device 10 in proximity with the lead 68 and the metallic coating 70. The battery 66 provides electric current to the metallic layer 70 so that the microbes or pathogens in the fluid path adjacent to the interior surface 72 do not attach to the surface 72 or the metallic coating 70. The electric current need not be strong enough to kill a pathogen; it need only repel the pathogen or a protein from residing on and subsequently forming a harmful biofilm on the metallic coating 70. In an alternate embodiment, the metallic coating 70 is not present. Rather, the lead 68 transfers electric current to the solution that is in contact with the interior surface 72. The solution then provides an environment that repels or is otherwise undesirable for the presence of a pathogen or protein and subsequent formation of a harmful biofilm.

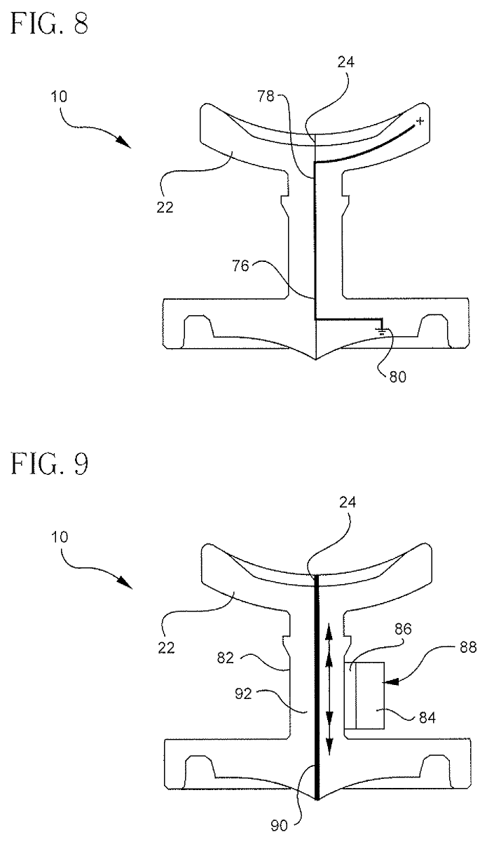

Referring now to FIG. 8, a cross section view of a septum 22 of a vascular access device 10 is shown with a heat conductor, heating element, or other electrically resistive film heater 76 located on, within, or adjacent to an interior surface 78 of the device 10. The heat conductor 76 may reside within, on, or near the interior surface 78 of the septum 22 or any other portion of the device 10. An energy source such as a battery 80 provides energy to the electrically resistive film heater 76 causing the heater 76 to heat to a level that is harmful or deadly to a pathogen. The body of the septum 22 may be formed of silicone or other material capable of withstanding very high temperatures including those up to 500° to 600° Fahrenheit. Thus, the heat conductor 76 is capable of heating to a harmful temperature for the pathogen without causing harm or damage to the material of the septum 22 or other portion of the device 10. In use, the battery 80 may transfer energy to the heat conductor 76 periodically and automatically, manually when initiated by an operator, and/or automatically upon screwing on an external power source on an exterior surface of the device 10. When the external power source is attached to the device 10, the heat conductor 76 may then begin to function under either an automatic or manual program as desired by the operator.

Referring now to FIG. 9, a cross section view of a septum 22 of a vascular access device 10 is shown. The septum 22 includes an oscillator 88 attached to an exterior surface 82 of the septum 22 in the form of a mass 84 attached to a piezo electric element 86. The oscillator 88 causes a rapid repetitive movement against the exterior surface 82 causing an interior surface 90 of the septum 22 to rapidly vibrate and move against an opposing side 92 of the septum 22. When the interior surface 90 vibrates rapidly against the opposing side 92, heat and friction is created within the slit 24 of the septum 22, thus repressing pathogenic activity within the slit 24 by killing the bacteria that reside therein.

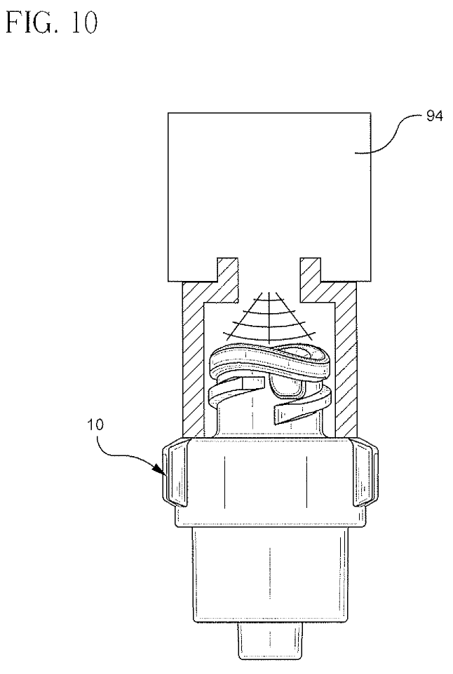

Referring now to FIG. 10, a vascular access device 10 may include or otherwise be coupled with a handheld wave generator 94. The wave generator 94 may be placed over the top of the vascular access device and an operator may turn the generator 94 on to initiate microwaves or percussion waves in the direction of the device 10. The microwaves or percussion waves are generated in accordance with a specific cycle including pulse frequency, wavelength, amplitude, period, and duration. Microwaves may be used to excite a bacterial or other pathogenic cell, causing the cell to overheat and die. Percussion waves may be used to shear a bacterial or other pathogenic cell apart from itself or from other neighboring harmful agents. The embodiment described with reference to FIG. 10 thus provides an energy source that is a wave generator capable of repressing a pathogen.

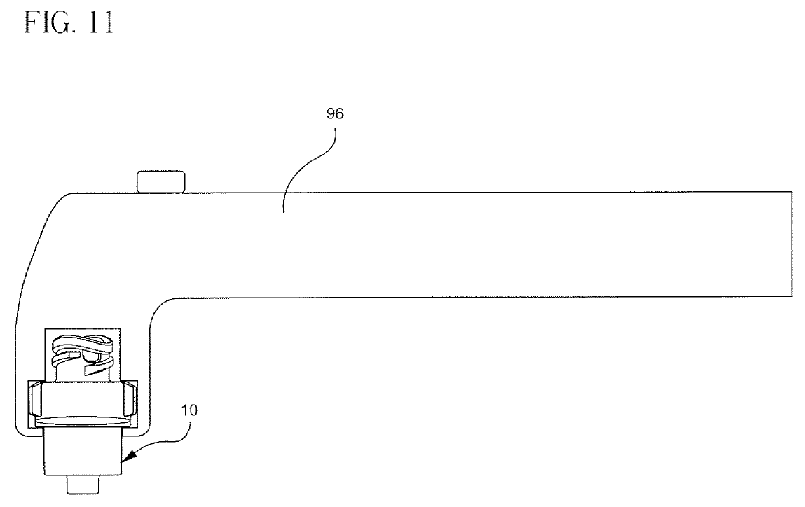

Referring now to FIG. 11, a vascular access device 10 is held on its exterior surface by an ultrasonic wave generator 96. An operator may use the ultrasonic wave generator 96 as an energy source to kill any bacteria within the device 10 and break up any biofilm that has formed on an interior surface of the device 10. The ultrasonic wave generator 96 may also be permanently attached to the device 10 in order to shake and kill any pathogen located within the device 10.

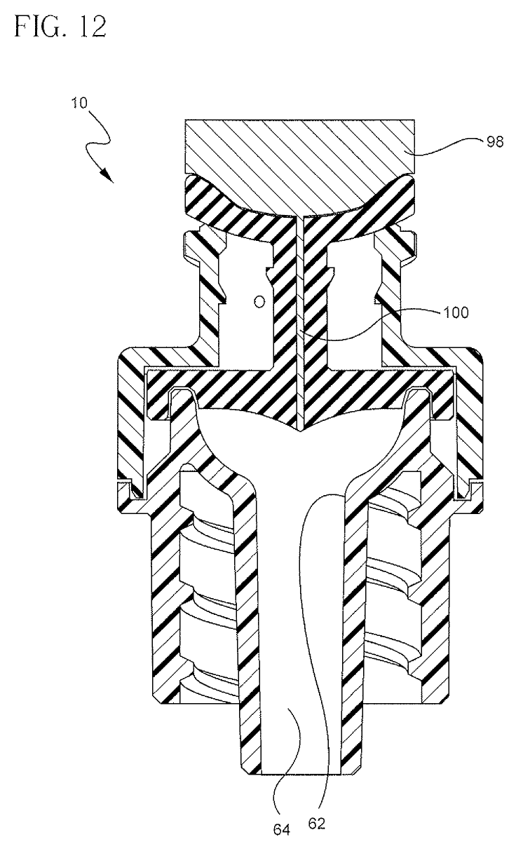

Referring now to FIG. 12, a cross section of a vascular access device 10 shows a medicated blade plug 98 that operates as an antiseptic applicator. The antiseptic applicator is a source of chemical energy capable of repressing a pathogen residing on an interior surface 100 of the device 10. The plug 98 may be inserted and retracted by an operator as needed or desired during use of the device 10.

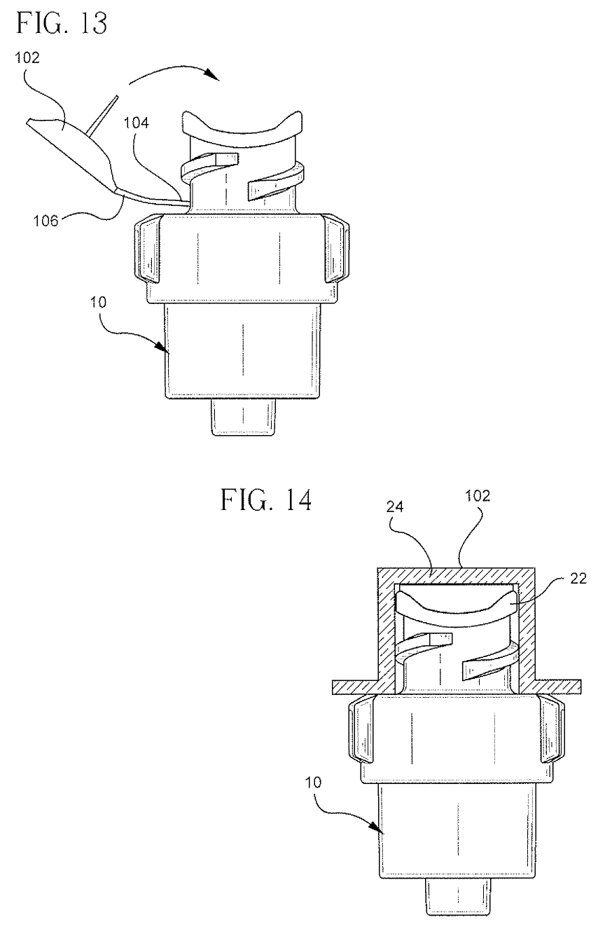

Referring now to FIG. 13, a side view of a vascular access device 10 shows a snap cap 102 integrated on a top surface 104 of the device 10. The snap cap 102 includes a medicated blade plug as described with reference to FIG. 12. The snap cap 102 also pivots upon a hinge 106 attached to the top surface 104 of the device 10.

Referring now to FIG. 14, the vascular access device 10 of FIG. 13 is shown with the snap cap 102 in closed position. When the snap cap 102 is in closed position, the medicated blade plug is inserted into the slit 24 of the septum 22 of the device 10 such that the medicated plug contacts any interior surface of the device 10 that is likely to have a pathogen residing thereon. The medicated surface or antiseptic of the medicated pad or plug will kill the pathogen. The medicated plug described with reference to FIGS. 13 and 14 may include a top pad 108 (shown in FIG. 13) that provides a saturated reservoir of medication or other antiseptic which may wick or otherwise travel down the length of the medicated plug and ultimately against an interior surface of the device 10.

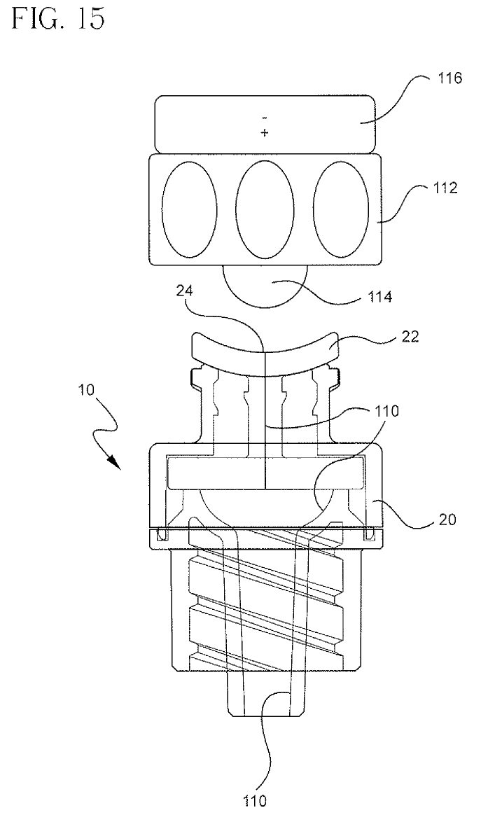

Referring now to FIG. 15, a vascular access device 10 housing a pathogen within its body 20 may be cleansed by means of an energy source that emits ultraviolet light on any portion of the interior surface 110 of the body 20. The energy source that emits ultraviolet light may be a cap 112 with an ultraviolet LED bulb in the shape of a male Luer or tip 30 of a separate device 26 (FIG. 1). The cap 112 includes an ultraviolet LED bulb 114 powered by a battery 116. The bulb 114 is turned on when the cap 112 is attached to the device 10. The bulb 114 may be turned on either manually by an operator or automatically as a result of the action of connecting the cap 112 with the device 10. For example, as the cap 112 is screwed onto the threads of the device 10, two contacts connecting the battery 116 with the bulb 114 may come into alignment causing the circuit between the bulb 114 and the battery 116 to be complete and the bulb 114 to be illuminated.

The ultraviolet LED bulb 114 of the cap 112 may operate for an intensity and duration necessary to repress a pathogen within the device 10. In an alternate embodiment, an ultraviolet LED bulb shines through the septum 22 of the device 10 without penetrating the slit 24 of the septum 22. In another embodiment, an ultraviolet LED bulb emits ultraviolet light through the housing 20 of the device 10 without penetrating the housing 20 or the slit 24 of the septum 22.

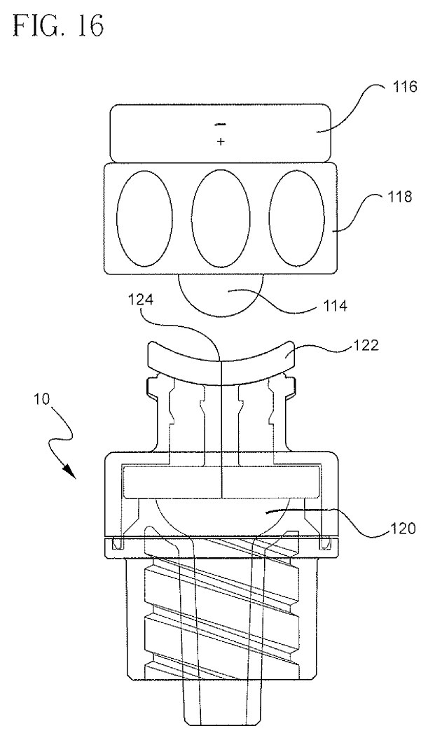

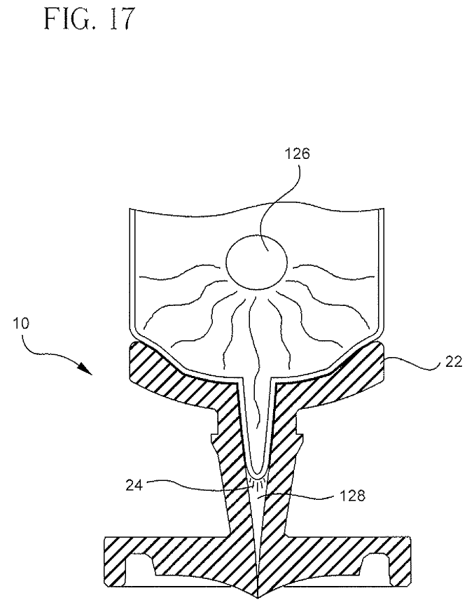

Referring now to FIG. 16, the embodiment described with reference to FIG. 15 may be modified with other solutions or structures to provide an energy source that employs ultraviolet light to repress a pathogen. In the alternate embodiment shown in FIG. 16, a vascular access device containing a pathogen may be sterilized using an ultraviolet light source 118 and a flush solution 120 that is infused into the device 10. The flush solution 120 is designed to optimally transmit the ultraviolet light from the light source 118 through the flush solution 120 to every interior surface 122 of the device 10. Additionally or alternatively, the embodiment described with reference to FIG. 16 may include an intravenous catheter or other structure 124 that transmits the ultraviolet light from the light source 118 down the length of the catheter to provide reflective or fluorescent emission of the ultraviolet light against all portions of the interior surface 122. In this manner, the various embodiments described with reference to FIG. 16 provide means of transmitting ultraviolet light within the device 10 to repress a pathogen.

Referring now to FIG. 17, an ultraviolet light energy source 126 is inserted into the slit 24 of a septum 22 of a vascular access device 10. The ultraviolet source 26 is a light pipe or custom molded LED casing that is shaped to fit within the slit 24 of the septum 22. The shape of the light source 126 permits the light source to directly emit ultraviolet light against an interior surface 128 of the device 10. The shape of the light source 126 may be modified as necessary to permit direct emission of ultraviolet light into and against any interior surface 128 of any vascular access device 10. Such a modification will provide an ultraviolet light source 126 capable of providing maximum pathogenic activity repression.

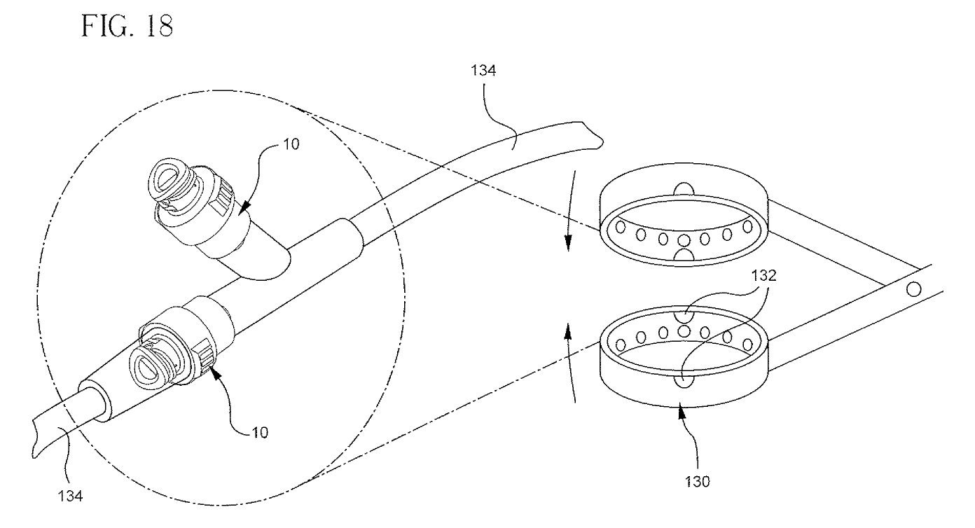

Referring now to FIG. 18, a vascular access device 10 may be completely encompassed or enshrouded by a handheld ultraviolet light isolator and exposer 130. The handheld ultraviolet light source 130 includes cut outs 132 necessary to fit around the extended tubing 134 of the device 10. When placed over the device 10, the handheld light source 130 will provide an isolated environment providing high intensity ultraviolet light to only an area on and within the vascular access device 10 that is likely to include a pathogen. When operated, the handheld light source 130 will provide sufficient ultraviolet light to the pathogen to repress its activity.

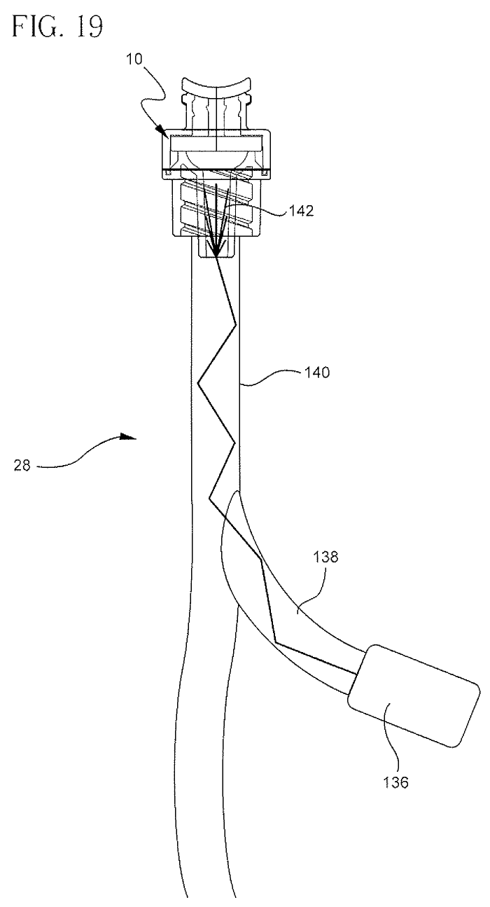

Referring now to FIG. 19, a vascular access device 10 may be attached to an ultraviolet light energy source 136. The ultraviolet light source 136 is attached downstream in the fluid path of the extravascular system 28 to which the device 10 is attached. For example, the light source 136 may be attached to an alternate pathway 138 of a catheter 140 to which the device 10 is connected. The ultraviolet light source 136 will then emit ultraviolet light through the alternate pathway 138 into the main body of the catheter 140 and ultimately into and against an interior surface 142 of the device 10. Along the entire length of the path of the ultraviolet light between the light source 136 and the device 10, the ultraviolet light will repress any pathogen that resides either within the device 10 and/or the catheter 140. The light source 136 can be periodically activated and/or turned off during administration of any substance, fluid, or other drug through the extravascular system 28.

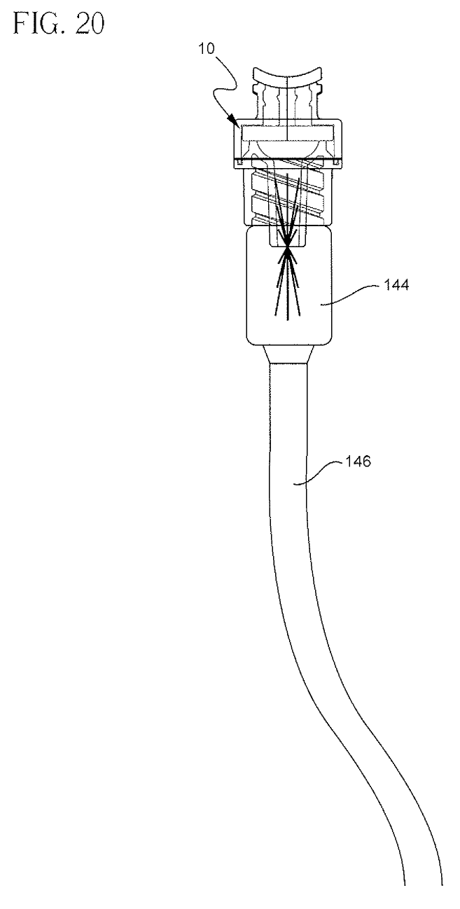

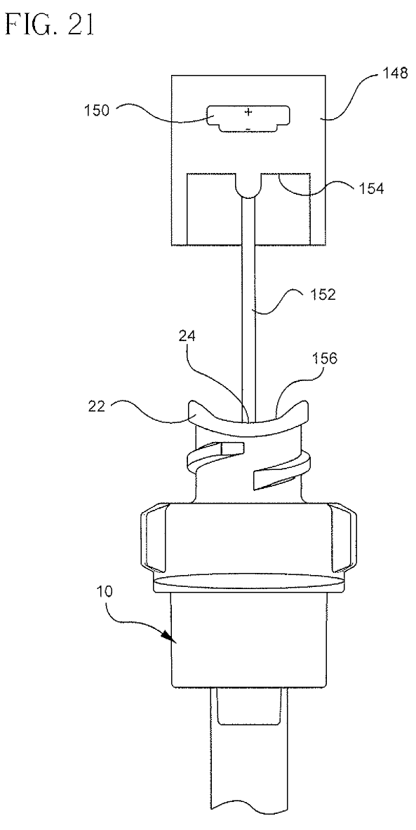

Referring now to FIG. 20, an alternate embodiment of the embodiment described with reference to FIG. 19 is shown. In this embodiment, an ultraviolet light source 144 is attached in series with and directly to the vascular access device 10. Thus, a bottom portion of the vascular access device 10 is attached to an upper portion of the light source 144, and a lower portion of the light source 144 is attached to a catheter 146. The light source 144 of FIG. 20 represses pathogens in a manner similar to the light source 136 of FIG. 19.

Referring now to FIG. 21, a vascular access device 10 is sterilized on its interior surface by a sterilization cap 148. The sterilization cap 148 is an embodiment which combines many of the features of previous embodiments, for example, the embodiments described with reference to FIGS. 12 through 17. As with the present embodiment, the features of any embodiment described herein may be combined with any of the features of any other embodiment described herein to produce an energy source capable of repressing a pathogen consistent with the principals of the present invention.

As shown in FIG. 21, the sterilization cap 148 includes a battery 150 that provides power to a fiber optic rod 152. The fiber optic rod 152 provides ultraviolet light to an interior surface of the device 10. The surface of the fiber optic rod 152 is abraded in a manner which permits the fiber optic rod 152 to emit ultraviolet light in an outward direction against an interior surface of the device 10. The sterilization cap 148 may also include a medication or antiseptic 154 on its interior surface. When the sterilization cap 148 is fully engaged with the vascular access device 10, the fiber optic rod 152 is inserted into and near or against the interior surface of the device 10 and the interior surface 154 of the cap 148 is placed in direct contact with a top surface 156 of the device 10. The rod 152 then sterilizes the interior surface of the device while the antiseptic 154 sterilizes the top surface 156 of the device 10. The sterilization cap 148 is either turned on manually by an operator or automatically as a result of the engagement of the cap 148 with the device 10.

The sterilization cap 148 may emit ultraviolet light when fully engaged with the device 10 only for a period of time necessary to repress a pathogen within the device 10. After the light is emitted for the necessary period of time, the sterilization cap 148 will cease emitting light within the device 10. However, since the cap 148 remains engaged with the device 10, the antiseptic 154 will continue to protect and sterilize the top surface 156 and the slit 24 of the septum 22 of the device 10 to inhibit the entry of any pathogen into the device 10 while the sterilization cap 148 is engaged with the device 10. When an operator is ready to later use the device 10 to infuse fluid into a patient, and/or draw blood from a patient, the operator removes the sterilization cap 148 from the device 10.

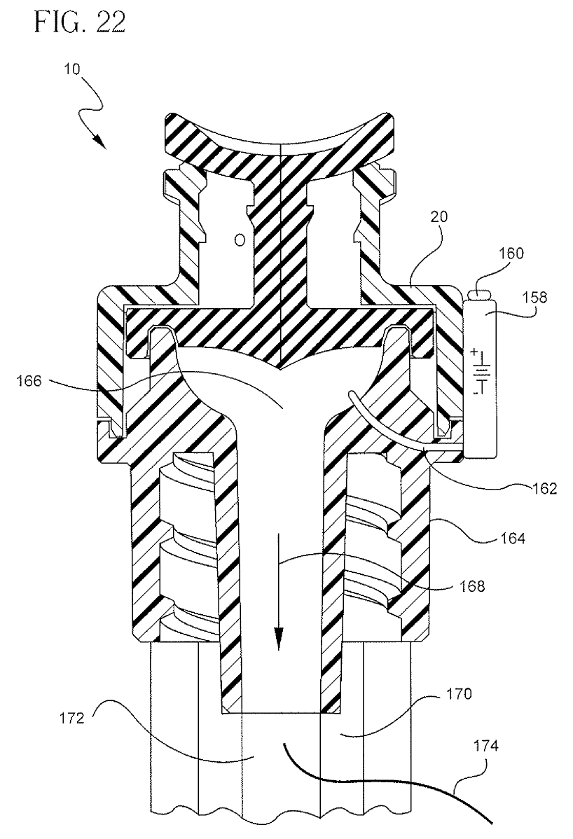

Referring now to FIG. 22, a vascular access device 10 includes a battery 158 secured to the body 20 of the vascular access device 10. The battery 158 includes a button 160, which an operator may press or actuate to activate operation of the battery 158. In operation, the battery 158 sends a current through a lead 162 from an exterior surface 164 of the device 10 to an interior cavity 166. The electric current then travels through the fluid housed within the interior cavity 166 in a direction 168 along the length of the device 10 and into an adjoining catheter 170. The electric current then travels from the interior cavity 172 of the catheter 170 to a grounding wire 174. The grounding wire 174 then carries the current away from the interior cavity 172 to a ground outside the device 10. Alternatively, the grounding wire 174 returns the electric current to the battery 158 to preserve its charge for future use.

In use, a sufficient amount of electric current is transferred from the battery 158 through the fluid of both internal cavities 166 and 172 in order to repress a pathogen. The grounding wire 174 is preferably located between the battery 158 and a patient and is situated in a manner to protect a patient from receiving any of the electric current from the battery 158 into the patient's vascular system. An operator may actuate the button 160 to release the electric current from the battery into the device 10 at any preferable time during the use of the device 10.

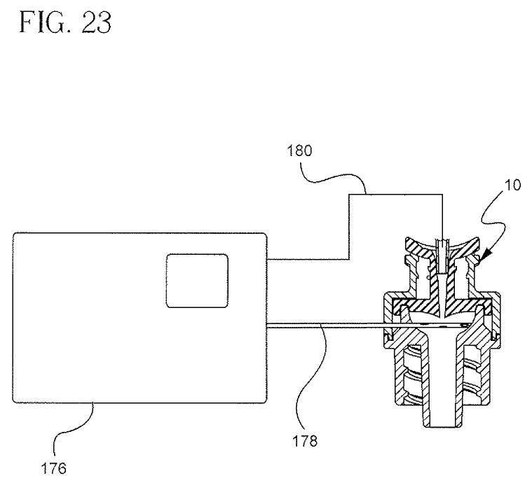

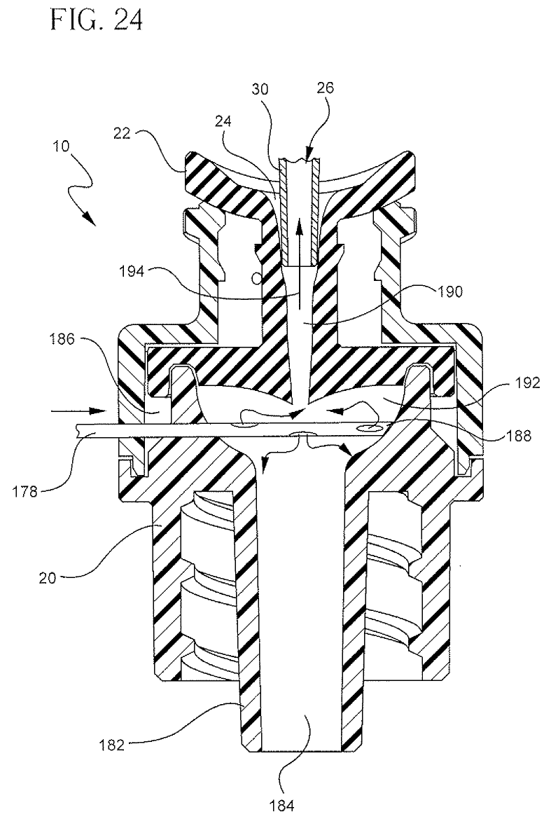

Referring now to FIG. 23, a flush pressure unit 176 is attached to a vascular access device 10. The flush pressure unit 176 rapidly transfers fluid into and out of the device 10 by means of an inlet flow path 178 and outlet flow path 180 in either a forward or reverse direction. The flush pressure unit 176 may infuse any antibacterial fluid including chlorine. The direction of the flow into and out of the device 10 may be oscillated in order to provide a preferably operation capable of repressing pathogenic activity. When received by the flush pressure unit 176, the fluid from the device 10 may be collected and later evaluated or otherwise analyzed to determine whether the device 10 has been colonized by a pathogen. Under analysis, the characteristics of the pathogen may be determined, and appropriate treatment to the device 10 and/or patient to which the device 10 is or was attached may be administered based on the results of the fluid evaluation.

Referring now to FIG. 24, the vascular access device 10 of FIG. 23 is shown in cross section view. The inlet fluid valve 178 includes a flush tip 182 that is inserted into a lower portion of the body 20 of the device 10. The flush tip 182 seals off a lower interior chamber 184 of the device 10. The flush tip 182 enters through the body 20 of the device 10 by penetrating a seal 186 which hinges open when the flush tip 182 is inserted. When the flush tip 182 is removed, the seal 186 resumes its original position forming a wall that is continuous with the body 20 of the device 10 in a manner that prevents fluid from escaping the device 10 through the seal 186 and permits the device 10 to undergo normal operation.

The flush tip 182 includes infusion pores 188 on its surface. Antimicrobial or other antibacterial fluid is flushed through the infusion pores 188 into an interior chamber 190 of the device 10. The pores 188 are situated at various strategic locations on the surface of the flush tip 182 to permit the fluid that is infused from the pores 188 to be rapidly ejected in a variety of directions against all interior surfaces 192 of the device 10. As the fluid is injected rapidly against all interior surfaces 192, the surfaces 192 are cleansed and any pathogen residing thereon is repressed. The fluid then carries the pathogen and other harmful materials in an upward direction 194 into the tip 30 of a separate device 26 or through the outlet fluid path 180 shown in FIG. 23. The direction 194 of the fluid may be reversed such that fluid is infused through the regular access port or slit 24 of the septum 22 and later removed through the seal 186. Saline and/or any other fluid may be pushed into or pulled from the interior chamber 190 through any number of pores within the tip 30, flush tip 182, and/or body 20 of the device 10.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

1. A medical device, comprising:
a vascular access device including a body and an interior surface of the body, wherein the interior surface communicates with a fluid capable of delivering a pathogen to the interior surface, and
an energy source coupled with the vascular access device, wherein the energy source provides energy to the interior surface of the body sufficient to repress pathogenic activity.

2. The medical device of claim 1, further comprising:
an interior structure in contact with the interior surface of the body, an exterior surface of the body, and
an exterior magnet in contact with the exterior surface of the body,
wherein the energy source is a magnetic force between the exterior magnet and the interior structure, and
wherein the movement of the exterior magnet causes movement of the interior structure.

3. The medical device of claim 1, wherein the interior surface of the body is formed from a degradable biocompatible material.

4. The medical device of claim 1, wherein the interior surface is formed from an electrically conductive material, and wherein the energy source is a battery that delivers electric current to the electrically conductive material.

5. The medical device of claim 1, wherein the interior surface includes a heat conductor, and wherein the energy source transfers heat to the heat conductor.

6. The medical device of claim 1, wherein the energy source is an oscillator that causes rapid repetitive movement of the interior surface.

7. The medical device of claim 1, wherein the energy source is a wave generator.

8. The medical device of claim 1, wherein the energy source is an antiseptic applicator.

9. The medical device of claim 1, wherein the energy source emits ultraviolet light on the interior surface of the body.

10. The medical device of claim 1, wherein the energy source delivers electric current to the fluid of sufficient magnitude and duration to repress pathogenic activity.

11. The medical device of claim 1, wherein the energy source includes anti-bacterial fluid applied to the interior surface of the body.

12. A method of repressing pathogenic activity in a vascular access device, comprising:
providing a vascular access device with a body having an interior surface, and
energizing the vascular access device to repress pathogenic activity on the interior surface.

13. The method of claim 12, wherein energizing includes actuating a magnet to disturb a pathogen residing on the interior surface.

14. The method of claim 12, wherein energizing includes degrading the interior surface.

15. The method of claim 12, wherein energizing includes supplying electric current to the interior surface.

16. The method of claim 12, wherein the interior surface includes a heat conductor and wherein energizing includes heating a heat conductor.

17. The method of claim 12, wherein energizing includes vibrating the interior surface.

18. The method of claim 12, wherein energizing includes generating and transmitting a series of waves against the interior surface.

19. The method of claim 12, wherein energizing includes sterilizing the interior surface.

20. The method of claim 12, wherein energizing includes emitting ultraviolet light towards the interior surface.

21. The method of claim 12, wherein energizing includes flushing the vascular access device with anti-bacterial fluid.

22. A medical device, comprising:
means for accessing the vascular system of a patient, and
means for repressing a pathogen, wherein the pathogen resides within the means for accessing the vascular system of a patient and wherein said means for repressing comprises an energy source.

23. The medical device of claim 22, wherein the means for accessing comprises:
a vascular access device including a body and an interior surface of the body, wherein the interior surface communicates with a fluid capable of delivering a pathogen to the interior surface, and
wherein the energy source is coupled with the vascular access device, wherein the energy source provides energy to the interior surface of the body sufficient to repress pathogenic activity.

24. The medical device of claim 23, further comprising:
an interior structure in contact with the interior surface of the body,
an exterior surface of the body, and
an exterior magnet in contact with the exterior surface of the body,
wherein the energy source is a magnetic force between the exterior magnet and the interior structure, and
wherein the movement of the exterior magnet causes movement of the interior structure.

25. The medical device of claim 23, wherein the interior surface of the body is formed from a degradable biocompatible material.

26. The medical device of claim 23, wherein the interior surface is formed from an electrically conductive material, and wherein the energy source is a battery that delivers electric current to the electrically conductive material.

27. The medical device of claim 23, wherein the interior surface includes a heat conductor, and wherein the energy source transfers heat to the heat conductor.

28. The medical device of claim 23, wherein the energy source is an oscillator that causes rapid repetitive movement of the interior surface.

29. The medical device of claim 23, wherein the energy source is a wave generator.

30. The medical device of claim 23, wherein the energy source is an antiseptic applicator.

31. The medical device of claim 23, wherein the energy source emits ultraviolet light on the interior surface of the body.

32. The medical device of claim 23, wherein the energy source delivers electric current to the fluid of sufficient magnitude and duration to repress pathogenic activity.

33. The medical device of claim 23, wherein the energy source includes anti-bacterial fluid applied to the interior surface of the body.
